(19) United States
(12) Patent Application Publication
(10) Pub. No.: US 2022/0347035 A1
(43) Pub. Date: Nov. 3, 2022

(54) Mandible Clamping Device

(71) Applicant: Chris Coleman, Hastings, FL (US)

(72) Inventor: Chris Coleman, Hastings, FL (US)

(21) Appl. No.: 17/243,635

(22) Filed: Apr. 29, 2021

(51) Int. Cl. A61G 99/00

(52) U.S. Cl. CPC A61G 99/00

(57) ABSTRACT

The positioning of the mandible clamp within the person's jaw or mouth is accomplished by either of two embodiments. One embodiment uses a pair of flanged surface that allow a person to push a barb through the gap in a person's teeth on the upper teeth and the lower teeth to hold the jaw shut during presentation to the family. This first embodiment contemplates use for a period who has natural teeth at the time of death. The second embodiment resembles a clamp that is placed over the person's gums both on the upper gum as well as the lower gum to secure the jaws together. This second embodiment is used with person's who did not have natural teeth at the time of death. After the respective openings are placed over the gum areas of the person's upper and lower gums a tension member slides over the outside surface of the clamp to produce gripping of the gum by the device. A plurality of gripping knobs on the interior of the respective clamps is provided to produce additional grip on the person's gums.

This is a continuation in part of a prior filed application with application Ser. No. 16/265,156 and a filing date of Feb. 1, 2019. The applicant is claiming priority of this application with the prior filed application.

### FIELD OF THE INVENTION

This device teaches a device to clamp the jaw together for presentation in a funeral setting. It is customary, whenever someone passes away, in order to maintain the integrity of the body and to make an appropriate appearance for the family and loved ones of the deceased. The jaw and mouth are closed to prevent an opening in the mouth of the deceased. Additionally there should be respect for the body and the desire to minimize mutilation of the body should also be contemplated. This device, which consists of two embodiments, is a simple, easy way to make sure that the mandible and the maxilla positions (lower and upper jaw bones) and shut and the lips shield the device from the family members.

### PRIOR ART

There are other prior art references in the prior art that discuss devices that hold the jaw open or closed. Most of the prior art discuss patents that are in the dental field and a representative example of this can be found at Young, U.S. Pat. No. 5,605,456, which is a dental articulator. This device has a different function than the current application. Another reference that also pertains to the dental filed can be found at Page, U.S. Pat. No. 2,968,868 is a mandible clamp but this clamp has a completely different structure.

### BRIEF SUMMARY OF THE INVENTION

Whenever a person passes away, in order to appropriately present the body for the loved ones, among other things, the funeral personnel will clamp the jawbones together so that there is no opening in the mouth that is seen by the loved ones of the deceased. In the prior art a specific gun and the use of wires has been used to tape the person's mouth shut. Another method that is found in the prior art is by using string that is strung through the nasal passages to clamp the jaw bones together. Neither of the prior art references expose the attachment method to the family. However, the prior art method does deface the individual and is time consuming.

The use of the gun and the stringing method, however, may lead to some mutilation of a body which is to be minimized at all costs.

This device has two embodiments. One is intended to be used for those persons who dies with natural teeth and the second is to be used with people who die without teeth. The stated purpose of either embodiments is to secure the jaw bones together and to make sure that the jaw bones stay shut during the presentation to the family.

The first embodiment is used when the individual has teeth present. In this first embodiment a first flanged surface with a curved barb at the opposite end is placed in the gap that is present in the person's front teeth. This device is not inserted into the person's gum or bone. This first flanged surface is connected to a second flanged surface with a short rod; the second flanged surface with a corresponding barb that is positioned at the opposite end of the flanged surface is placed in the gap between the teeth on the lower jaw. The flanged surface is pressed into the person's mouth and abuts the teeth of the person. The gums and lips of the person will hide this device from the family during the viewing. This device will remain with the person.

The second embodiment is designed for people who die without natural teeth. In this embodiment the device resembles a C-clamp that can be locked in place with the use of another arcuate portion that fits over the C-clamp to tighten the device around the gum structure of the person. The C-clamp will have several protrusions that will prevent the device from dislodging during the viewing. One C-clamp is placed over the gum of the upper jaw and the second C-clamp is placed over the gum structure of the lower job. The lower jaw is then pressed gently to close the jaws of the person. The gums and lips of the person will hide this device from view by the family. This device, like the first device, remains with the person.

### BRIEF DESCRIPTION OF THE DRAWINGS

FIG. 1 is an isometric view of the first embodiment.

FIG. 2 is a side view of the first embodiment showing the flanged surfaces and the barbs.

FIG. 3 is a top view of the first embodiment.

FIG. 4 is an isometric view of the second embodiment.

FIG. 5 is a reverse isometric view of the second embodiment.

FIG. 6 is a side view of the second embodiment.

FIG. 7 is a top view of the second embodiment.

FIG. 8 is an in-use view of the first embodiment.

### NUMBERING REFERENCES

1 First Embodiment
5 Flanged surface
10 Barb
15 Stem
20 Connecting Bar
50 Second Embodiment
55 Clamp
60 Tension Member
61 C-clamp top surface opening
62 Tension member locking mechanism
63 C-clamp opening
65 Interior Protrusions

### DETAILED DESCRIPTION OF THE EMBODIMENTS

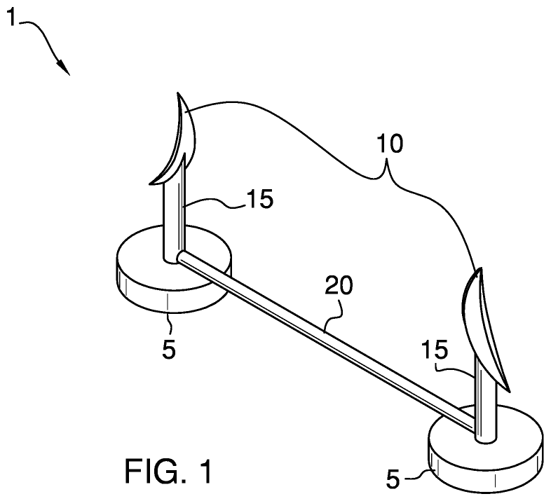

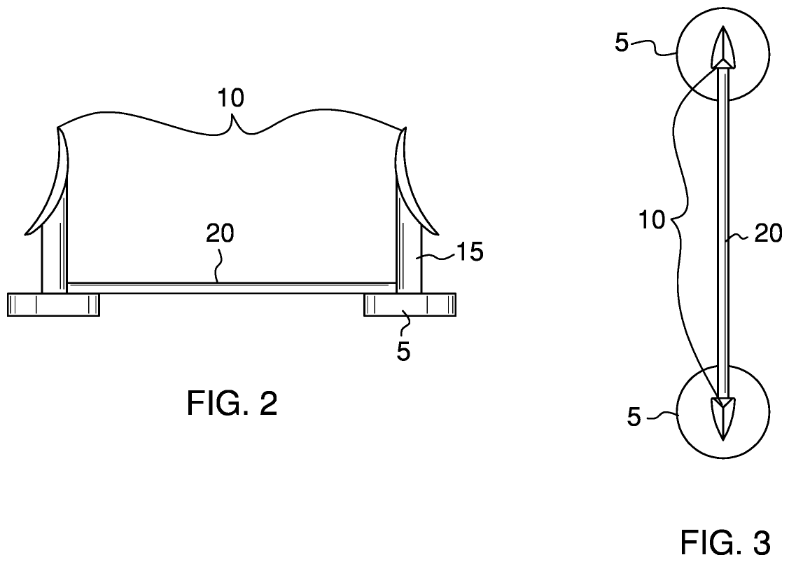

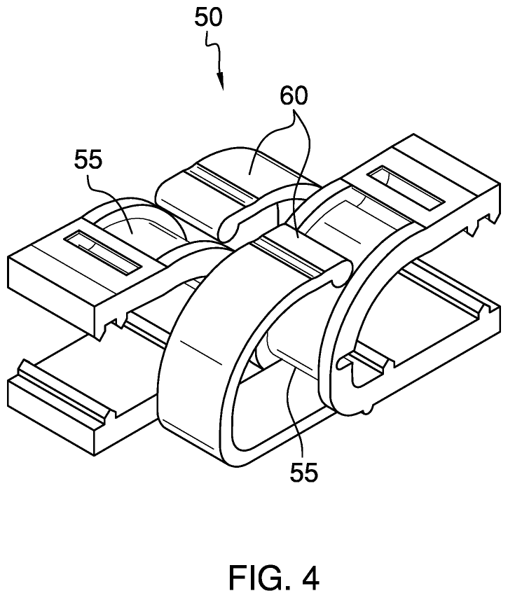

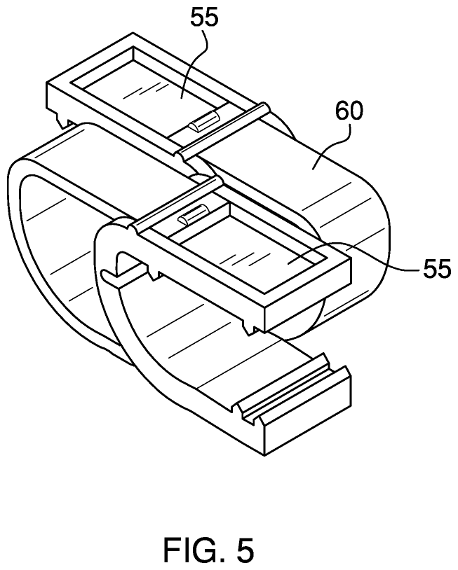

Two separate embodiments are taught in this application although they both teach a device that will make sure that the jaws of someone who has passed away do not open when that person is lying in state. The first of the embodiments is depicted in FIGS. 1-3 and the second embodiment is depicted in FIGS. 4-7. The first embodiment 1 is used with persons who die with natural teeth and the second embodiment 50 is used with people who die without natural teeth. Regardless of whether the person dies with or without teeth the function of either embodiment is still the same: keeping the person's jaw bones together to present to the family after that person has died.

Regardless of the embodiment the device will be covered by the person's gums and lips so that it is not seen by family members and loved ones; the device is intended to remain with the body.

### FIRST EMBODIMENT

Two flanged surfaces 5 are attached to a stem 15 with a curved barb 10 at each opposite end of the flanged surface. The pair of flanged surfaces 5 with attached stems 15 are connected to a curved barb 10 at the end of the respective stems.

The barb 10 of each end is placed in the gap in the teeth of the person's natural teeth. The flanged, flat Surface 5 is pressed so that the barbs are inserted into the interior of the person's mouth and the flanged surface abuts the person's teeth when the device is finally placed in the person's mouth.

Once the device has been installed in the person's mouth the respective barb 10 prevents the device from backing out and allowing the person's mouth to open.

Neither barb is inserted into the gum or jawbone of the person. The flanged surfaces are connected to each other with a connecting bar 20. A pair of stems 15 connect the flanged surface to the curved barb 10. The distance between the flanged surfaces is between three-quarters and one and one-half inches to accommodate differences in a person's mouth. Once the device is installed, it will remain hidden from view and will remain with the body.

After the body has been prepared and the mouth of the person has been closed, the person preparing the body lifts the bottom lip and top lip and aligns the barbs with the gaps between a person's teeth. The preparer then presses the flanged surface 5 to push the barb 10 between the gaps in the person's teeth. The preparer will permit the lips to fall back into their normal position and the device is hidden from view.

### SECOND EMBODIMENT

The second embodiment 50 teaches a mandible clamping device which is to be used with a person who dies without natural teeth. There are two clamps 55 which are joined together as one unit. The second embodiment is in the shape of a pair of C-clamps with the C-clamp openings 63 opposed to each other; the openings of the C-clamps are offset from each other. When the device is installed in the person's mouth the openings of the respective “Cs” are placed over the upper and lower gums of the person. The two sections of the device are connected together to ensure that the mouth of the person remains shut and the two sections remain joined together.

On the outside surface of the clamp 55 is another tension member 60 that slides over the outside of the clamp and causes the opening of the “C’ to clamp around the gums of the person. An top surface opening on the C-clamp 61 is provided to lock the tension member in position once it is placed on the person's gums. A tension member locking mechanism 62 is provided and will be placed in the top surface opening of the C-clamp 61 to insure that the tension member 60 does not slide backwards from the C-clamp.

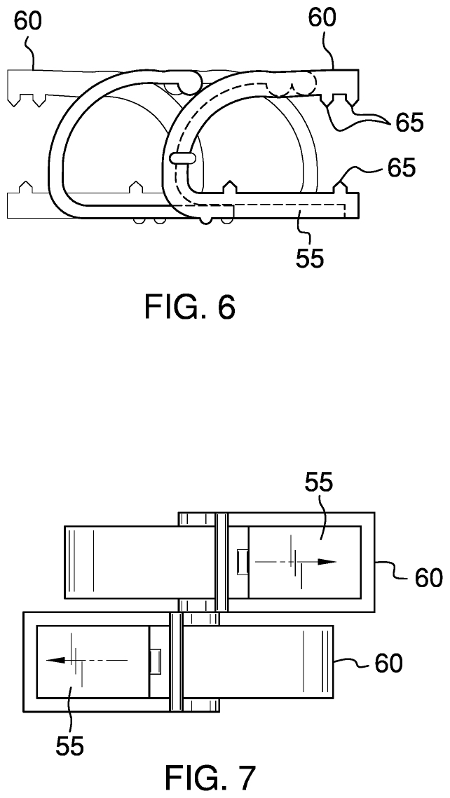

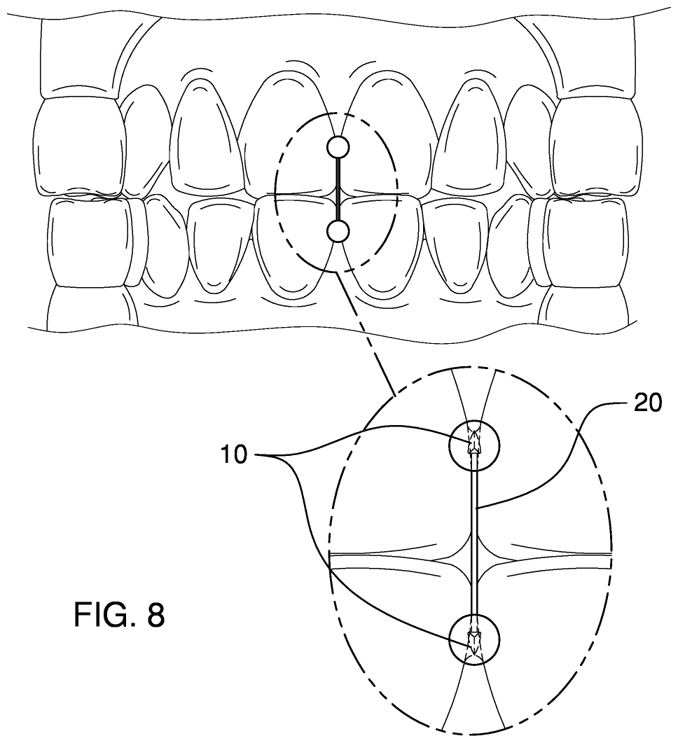

A plurality of interior protrusions 65 are placed in the interior space of the respective “Cs” such as depicted in FIG. 6 to provide additional gripping ability on the gums of the person.

After the body is prepared the person would open the mouth of the person and the first C-Clamp opening is placed over the upper jaw of the person. The preparer slides the tension member 60 over the outside of the C-clamp 55; the tension member locking mechanism will be inserted into the C-clamp top surface opening. Once the tension member slides over the outside surface of the C-Clamp the interior protrusions 65 will contact the gum surface of the person provide a snug fit over the person's gum. Once the first of the C-clamps is placed over the upper jaw the opposing or second C-Clamp is placed over the gum of the lower jaw. The preparer will slide the tension member over the outside surface of the C-clamp 55 and the protrusion that are in the inside of the C-Clamp will contact the person's gum surface on the lower jaw. Once both C-Clamps have been installed the prepare will then gently close the person's mouth so that the device is not visible. The gums and lips of the person will cover the device and will remain with the body.

1. A mandible clamping device, which is comprised of
a pair of flanged surfaces;
a pair of barbs;
wherein the barbs are curved;
a pair of stems;
wherein the stems connect the flanged surfaces to the barbs;
a connecting rod;
wherein the connecting rod connects the pair of flanged surfaces;
wherein the length of the connecting rod is in the range of three-fourth inch and one and a half inches.

2. A method to use the mandible clamping device as described in claim 1 which is comprised of the following steps:
a. moving the person's lips and gums to expose the person's teeth;
b. placing a barb in the gap in the person's teeth of the upper jaw;
c. placing a barb in the gap in the person's teeth of the lower jaw;
d. pressing the flanged surface to insert the barbs into the gaps in the person's teeth;
e. placing the person's lips and gums over the device.

3. A mandible clamping device, which is comprised of:
a pair of C-clamps;
wherein the C-Clamps are connected to each other;
wherein the openings of the C-clamps are opposed to each other;
wherein the openings of the C-clamps are offset from each other;
an opening on the top surface of the C-clamp;
a pair of tension members;
wherein a tension member is provided for each of the C-clamps;
wherein each of the tension members are placed on the outside surface of the C-clamp;
a tension member protrusion;
wherein the tension member protrusion will be inserted into the opening on the top surface of the C-clamp;
a plurality of interior protrusions;
wherein the plurality of interior protrusion are provided in the interior of the opening of the C-clamp;
wherein the tension member slides over the clamp.

4. A method to use the mandible clamping device as described in claim 1, which is comprised of the following steps:
a. opening the person's mouth
b. inserting the opening of the first C-clamp over the gum of the upper jaw;
c. sliding the tension member for the first C-clamp over the outer surface of the first C-clamp until the tension member protrusion slides into the opening of the first C-clamp;
d. inserting the opening of the second C-clamp over the gum of the lower jaw;
e. sliding the tension member of the second C-clamp over the outer surface of the second C-clamp until the tension member protrusion slides into the opening of the second C-clamp;
f. closing the person's mouth.
